(19) United States
(12) Patent Application Publication
(10) Pub. No.: US 2006/0271037 A1
(43) Pub. Date: Nov. 30, 2006

(54) Assisted systems and methods for performing transvaginal hysterectomies

(75) Inventors: John Maroney, Woodside, CA (US); Joseph Eder, Los Altos Hills, CA (US); Camran Nezhat, Woodside, CA (US)

Correspondence Address: GLENN PATENT GROUP, 3475 EDISON WAY, SUITE L, MENLO PARK, CA, 94025, US

(73) Assignee: Forcept, Inc., Menlo Park, CA (US)

(21) Appl. No.: 11/137,970

(22) Filed: May 25, 2005

(51) Int. Cl. A61B 18/14

(52) U.S. Cl. 606/45; 606/48; 606/49; 606/50

(57) ABSTRACT

Transvaginal hysterectomy is performed by mechanically engaging and extracting a uterus through opposed cutting and cauterizing elements. The cutting and cauterizing elements are typically provided on an expandable frame which may be positioned at the vaginal os. An associated traction device may be used to pull the uterus through the frame at a controlled rate.

### BACKGROUND OF THE INVENTION

1. Field of the Invention

The present invention relates generally to medical apparatus and methods. More particularly, the present invention relates to methods, systems, and devices for performing vaginal hysterectomy by extracting a uterus through opposed cutting and cauterizing elements.

A surgical procedure for removing a uterus is referred to is a “hysterectomy.” hysterectomy is most commonly performed via an abdominal incision but can also be performed via laparoscopy or transvaginally. The present invention is particularly concerned with transvaginal hysterectomy where the uterus is accessed through the vagina, the cervix is resected from surrounding tissues, and the uterus is removed through the vagina by manually pulling on the cervix and body of the uterus using forceps.

While transvaginal hysterectomy is advantageous in that it does not require a surgical incision or laparoscopic penetration, many physicians have difficulty with the procedure, particularly with control of bleeding (hemostasis).

In addition to being anchored at the cervix, the uterus is connected to surrounding tissues by ligaments and blood vessels which are generally located along a lateral plane through the uterus. After the cervix is mobilized, the cervix and body of the uterus are brought forward through the vagina, requiring dissection of the blood vessels and supporting ligaments as the uterus emerges. Typically, electrocautery scissors and similar devices are used to cut the blood vessels and stop bleeding. The need to simultaneously apply force manually to withdraw the uterus while cutting and cauterizing the blood vessels and other supporting ligaments and tissues can be quite challenging.

For these reasons, it would be desirable to provide improved methods, apparatus, and systems for performing transvaginal hysterectomy. It would be desirable if such improved methods, apparatus, and systems would generally mimic or duplicate the steps used in conventional transvaginal hysterectomy but minimize the manual and repetitive aspects of the procedure. In particular, it would be desirable to provide for improved dissection of blood vessels, ligaments, and other tissues attached to the outside of the uterus while providing for automatic or otherwise enhanced bleeding control while the uterus is being withdrawn. At least some of these objectives will be met by the inventions described herein below.

2. Description of Background Art

Methods for performing conventional transvaginal hysterectomy are described in Kovac (2004) Obstet. Gynecol. 103: 1321-1325. Patents describing laparoscopic and other hysterectomy procedures include U.S. Pat. Nos. 5,840,077; 5,662,676; 5,520,698; 5,116,327; 5,108,408; 5,041,101; 4,976,717; and 4,072,153.

### BRIEF SUMMARY OF THE INVENTION

According to the present invention, methods for transvaginal hysterectomy comprise gauging and extracting a uterus through a vagina at a controlled rate. The uterus is “mechanically” engaged, typically using an automatic traction device, and the uterus is passed through opposed cutting and cauterizing elements as it is extracted. The cutting and cauterizing elements are typically mounted or otherwise coupled to a frame, and energy is applied to the elements in order to cauterize blood vessels between the uterus and supporting tissue before the blood vessels are cut. The frame will typically be positioned near a vaginal opening or “os” but could also be configured to be positioned near the cervical os.

In preferred methods of the present invention, mechanically engaging and extracting the uterus will comprise first dissecting the cervix from surrounding tissue and then positioning the cervix in a traction device. The traction device is then operated to extract the uterus at the controlled rate. Preferably, the traction device will be coupled to the same frame as the opposed cutting and cauterizing elements. The traction device will thus be able to draw the uterus through the opposed cutting and cauterizing elements at the controlled rate in order to assure that the blood vessels are properly cauterized before they are cut. As used in present applications, the term “cauterize” will mean that the tissue is treated with energy to prevent or inhibit bleeding upon subsequent cutting. Typically, cauterization will be effected by the application of heat or electrical energy, more typically by applying radiofrequency (RF) energy through electrodes to induce ohmic heating within the tissue to achieve the desired hemostatis. Alternatively, energy can be applied to the tissue via electrical resistance heaters, ultrasonic transducers, microwave antennas or emitters, or in some cases through cryogenic (cooling) sources in order to induce the desired hemostatis. As described hereinafter, the methods and apparatus will preferably employ radiofrequency electrodes which may be monopolar or bipolar but in all cases will be arranged to induce ohmic heating within the tissue to cause the desired hemostatis.

It will sometimes be desirable to monitor the rate and/or force with which the traction device is extracting the uterus. Should a complication or a malfunction occur, it will be desirable to provide an alarm and/or stop operation of the traction device, typically when a preselected rate or force of extraction has been exceeded.

In some embodiments of the present invention, the cervix may be manually dissected and pulled in order to pass the uterus through the opposed cutting and cauterizing elements on the frame. In such cases, the use of an automatic traction device is not necessary. The opposed cutting and cauterizing elements will still provide facilitated cutting and cauterization the blood vessels, ligaments, and other tissue structures extending between the uterus and surrounding tissues. Additionally, the frame may provide rollers, optical elements, or other means for mechanically monitoring the rate and/or force of pulling in order to provide an alarm should the rate of extraction be excessive (thus compromising the ability of the frame to provide proper cauterization and hemostatis) or should the force of pulling be excessive, exposing the patient to risk.

Usually, the frame through which the uterus is extracted will be expandable to accommodate changes in the cross-sectional area of the uterus as it is advanced through the frame. Additionally, the cutting and cauterizing elements are usually formed separately and arranged sequentially so that the blood vessels are cauterized prior to cutting. Alternatively, however, it may be desired to provide integrated cutting and cauterizing elements so that the blood vessels are cauterized and cut by the same element. In the latter case, the elements are typically radiofrequency electrodes having regions suitable for coagulation as well as for cutting. By then energizing the electrodes at different times with a cutting current and/or coagulation current, the cauterization can be achieved prior to cutting.

In a second aspect, the present invention provides a system for assisting transvaginal hysterectomy. The system typically comprises a frame positionable adjacent a vaginal os or a cervical os. The frame defines an opening for passing a mobilized uterus therethrough and coagulation and cutting elements positioned relative to the opening to engage blood vessels, ligaments, and other structures which connect the uterus to supporting tissues as the mobilized uterus is drawn through the opening. Usually, the system further comprises a traction device coupled to the frame. The traction device is adapted to engage the mobilized uterus and advance the mobilized uterus through the opening. A traction device may comprise a variety of mechanical advancement mechanisms, such as reciprocating grippers, where the grippers typically have tissue engaging surfaces, such as toothed surfaces. Alternately, the traction device could comprise rotating elements, for example comprising rotating electrode surfaces which both apply energy to the uterine wall and advance the uterus through the opening.

The frame and/or the cutting and coagulation elements will typically be adjustably mounted to separate in order to accommodate the width of the uterus as it passes through the opening and frame. For example, the frame could define a split ring having halves which are biased closed and which will move apart as the uterus is drawn therethrough. In the later case, at least one cutting and coagulation element will typically be coupled to each half of the ring. The systems may further comprise a tissue dissection blade disposed across the frame opening, typically behind the coagulation and cutting elements so that the uterine body may be dissected after it has passed through the frame opening. Such dissection facilitates removal of the uterus. In still other embodiments, the electrodes may be cylinder culled and rotatively mounted on the frame.

### BRIEF DESCRIPTION OF THE DRAWINGS

FIG. 1 is a prospective view of a transvaginal hysterectomy system constructed in accordance with the principles of the present invention.

FIG. 2 is a cross-sectional view taken along line 2-2 of FIG. 1.

FIGS. 2A AND 2B are alternative cross-sectional views similar to FIG. 2.

FIG. 3 is a cross-sectional view taken along line 3-3 of FIG. 1.

FIGS. 4-6 illustrate use of the system of FIG. 1 for performing an assisted transvaginal hysterectomy protocol according to the methods of the present invention.

### DETAILED DESCRIPTION OF THE INVENTION

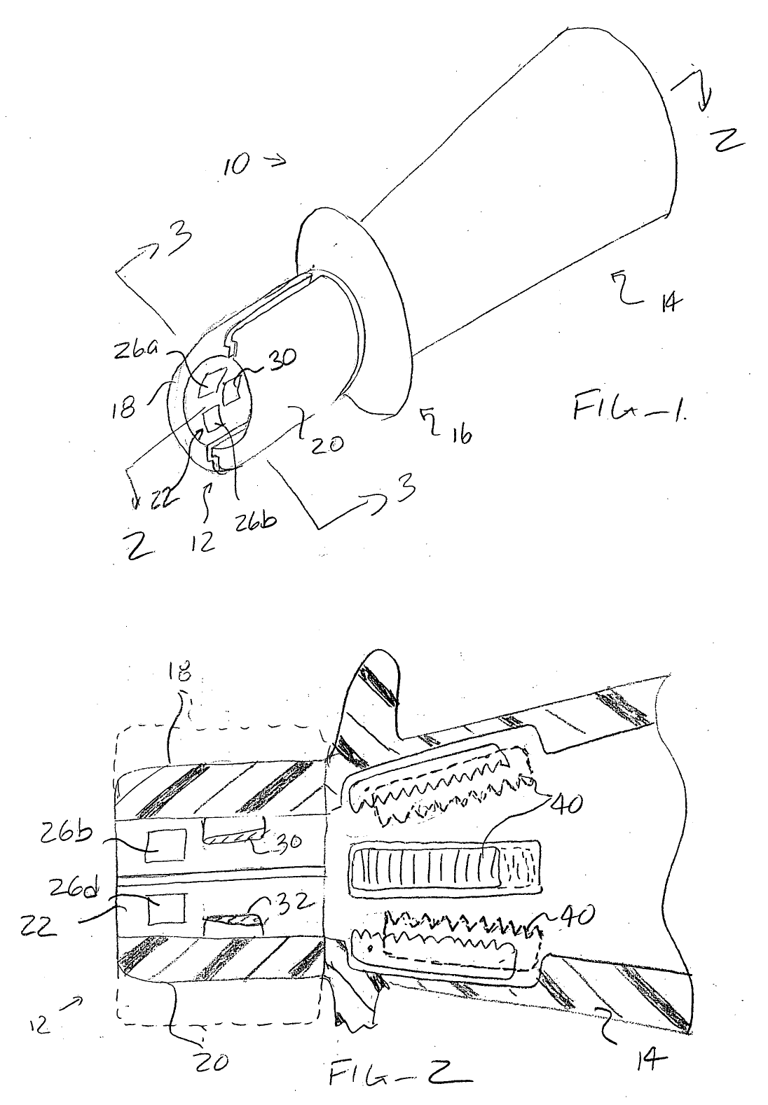

A transvaginal hysterectomy system 10 constructed in accordance with the principles of the present invention is illustrated in FIGS. 1-3. The system 10 includes a distal ring assembly 12, proximal body portion 14, and an insertion flange 16. The insertion flange will be used for positioning the device against the vaginal os when the distal ring assembly 12 is to be positioned within the vagina. In other embodiments where the device might be positioned outside of the vaginal os, the flange 16 would not be necessary.

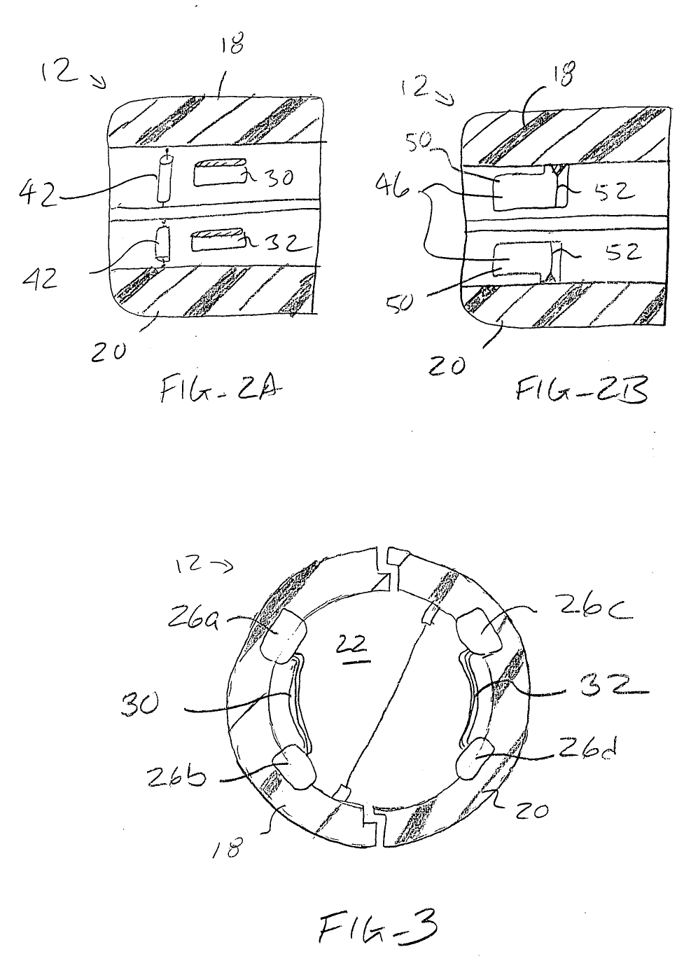

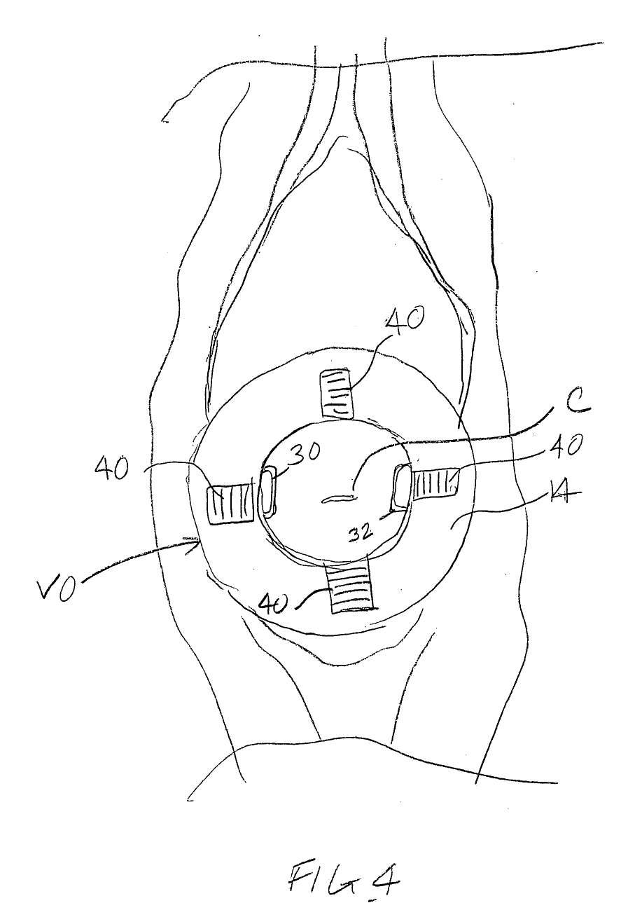

Referring now in particular to FIGS. 2 and 3, the distal ring assembly 12 comprises ring halves 18 and 20 which surround an opening 22. The ring halves 18 and 20 can move transversely apart, as shown in broken line in FIG. 2, and respond to passage of the uterus therethrough. Thus, the ring assembly 12 can accommodate different uterine sizes of different patients.

Four electrode pads 26a, b, c, and d are formed over an inner surface of the distal ring assembly, as best shown in FIG. 3. The electrode pads 26 are connectable to a radiofrequency power supply (not shown) which can deliver radiofrequency energy to the electrodes. In some cases, the electrodes 26a, b, c, and d can be operated in a monopolar mode where each of the electrodes is connected to one pole of the power supply while the second pole is connected to a dispersive pad (not shown) which is placed on the patient's back, thighs, or other conventional location. Alternatively, the electrode pairs 26a and b and 26c and d can be connected to opposite poles so that they operate in a bipolar fashion. In this way, the tissue between the pairs of electrodes 26a and 26b as well as 26c and 26d will be exposed to a relatively intense localized radiofrequency field in order to induce ohmic heating in the uterine tissue along the lateral edges of the uterus where the blood vessels, ligaments, and other supporting tissues are being dissected.

It will usually be desirable to mount the electrodes so that they will firmly engage tissue to provide good electrical contact with the tissue and preferably compress the tissue to enhance cauterization. Such mounting may comprise springs or other passive compliant structure and/or may include pistons, cages, levelers, positioners, and other active structures for inwardly compressing the electrodes against the tissue as the tissue passes through the frame.

Immediately proximal to the electrodes 16a, b, c, and d are a plurality of cutting blades or other cutting structures, typically a pair of cutting blades 30 and 32. Cutting blades will typically be conventional blades with honed edges for cutting through the blood vessels, ligaments, and other supporting tissues as the uterus is drawn through at opening 22 of the distal ring assembly 12. In some case, however, it may be desirable to connect the cutting blades to a radiofrequency power supply which can provide a desired cutting current to the blades to assist in cutting and optionally to provide also a cauterizing current to further assure cauterization of the blood vessels and other tissue structures.

A traction device comprising opposed reciprocating grippers 40 is provided in the proximal body portion 14 of the system 10. A driver (not shown) will be provided for mechanically reciprocating the grippers from a distal retracted position (shown in full line in FIG. 2) to a proximal radially inward position (shown in broken line in FIG. 2). By reciprocating the grippers 40 in this pattern, the uterus may be mechanically extracted and pulled through the opening 22 of ring assembly 12 in order to both coagulate and cut the blood vessels, ligaments and other supporting structures in a controlled manner.

Referring now to FIG. 2A, in place of the electrode pads 26, roller electrodes 42 may be provided in the distal ring assembly 12. The roller electrodes may be driven by a powered drive unit (not shown) to effect or assist traction of the uterus. Alternatively, the rollers may be passive, i.e., roll in response to passage of the uterus. Pairs of rollers could also be used to support “tread” structures which may be passive or be actively driven. Similarly, referring to FIG. 2B, in place of both the electrodes 26 and cutting blades 30 and 32, an electrode surgical electrode structure 46 may be provided in the distal ring assembly 12. The structure 46 will include an electrocautery portion at a distal end of the structure and an electrosurgical or cutting portion 52 at a proximal end of the structure. In this way, the electrosurgical power supply (not shown) can provide both the cauterizing current to the distal portion 50 and cutting current to the proximal portion 52 of the integrated electrode structure.

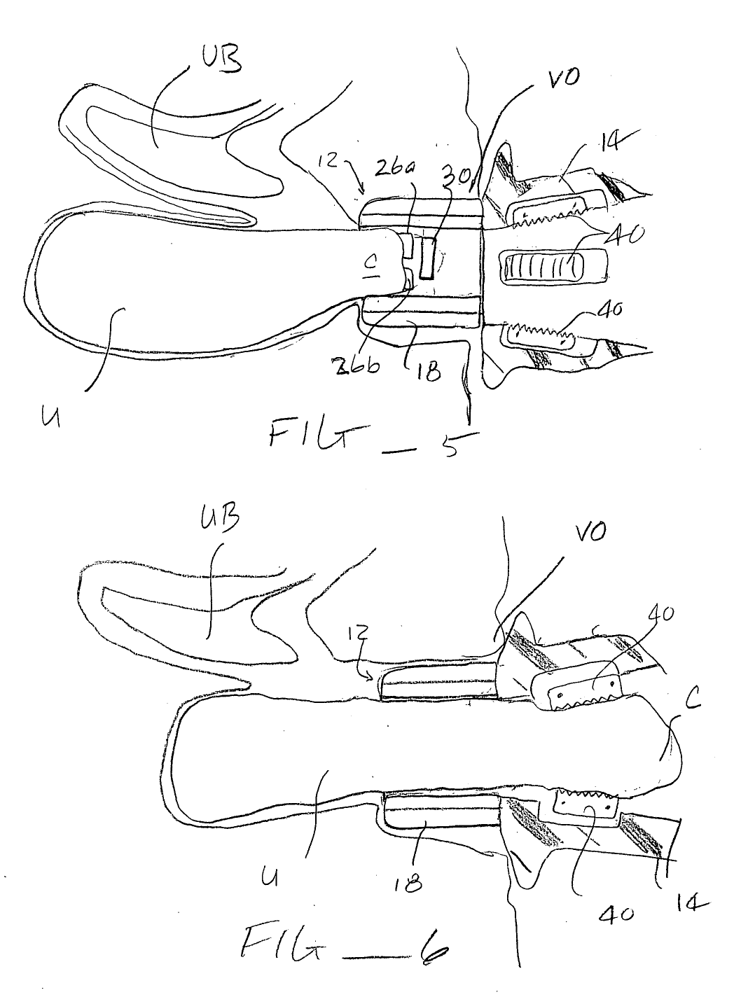

Referring now to FIGS. 4-6, use of the system 10 for extracting a uterus U according to methods of the present invention will be described. The system 10 is introduced so that the distal ring assembly 12 passes through the vaginal os exposing the cervix C through opening 22 in the ring assembly. The cervix C may be viewed by the physician through the proximal end of the body portion 14, as shown in FIG. 4. The cervix is then mobilized by dissecting from surrounding tissues, and the head of the cervix drawn into the ring assembly 12, as shown in FIG. 5. The main body of the uterus remains in place underneath the urinary bladder (UB), while the cervix passes through the electrodes 26 toward the cutting blades 30 and 32.

After the cervix is withdrawn into the distal and of the body portion 14, the reciprocating grippers 40 may be activated to engage and proximally advance the body of the uterus, as best shown in FIG. 6. The uterus will be advance at a rate of about 0.1 cm/min to about 10 cm/min, while the blood vessels, ligaments, and other tissue structures on each side of the uterus are first being cauterized by the electrode elements 26a, b, c, and d and subsequently dissected by the blades 30 and 32. Optionally, the force of extraction and/or the rate of extraction can be monitored and alarms provided or the system automatically shut down. After the uterus has been completely withdrawn, the hysterectomy procedure can be completed in a manner similar to that for conventional transvaginal hysterectomies.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for transvaginal hysterectomy, said method comprising:
mechanically engaging and extracting a uterus through a vagina at a controlled rate;
passing the uterus as it is extracted through a frame having opposed cutting and cauterizing elements and applying energy to the elements to cauterize blood vessels between the uterus and supporting tissue before they are cut.

2. A method as in claim 1, wherein the same is positioned near a vaginal os.

3. A method as in claim 3, wherein the same is positioned near the cervical os.

4. A method as in claim 1, wherein mechanically engaging and extracting the uterus comprises:
dissected the cervix from surrounding tissue;
positioning the cervix in a traction device coupled to the frame; and
operating the traction device to extract the uterus at the controlled rate.

5. A method as in claim 4, wherein the cervix is manually dissected and pulled forward to the traction device.

6. A method as in claim 2, further comprising monitoring rate and/or force with which the traction device extracts the uterus.

7. A method as in claim 6, further comprising providing an alarm and/or stopping operation of the traction device if a preselected rate or force of extraction is exceeded.

8. A method as in claim 1, wherein the same is expandable to accommodate changes in cross-sectional area of the uterus as it is passed through the frame.

9. A method as in claim 1, wherein the cutting and cauterizing elements a-c separate and arranged sequentially so that the blood vessels are cauterized prior to cutting.

10. A method as in claim 1, wherein the cutting and cauterizing elements a-c integrated so that the blood vessels a-c cauterized and cut by the same elements.

11. A method as in claim 1, wherein applying energy comprises applying a radiofrequency coagulation current to the blood vessels.

12. A method as in claim 1, wherein mechanically engaging and extracting the uterus comprises:
manually dissecting and pulling a cervix of the uterus to pass the uterus through the same.

13. A method as in claim 12, further comprising mechanically monitoring the rate and/or force of pulling.

14. A method as in claim 13, further comprising providing an alarm if the rate or force exceeds a preselected value.

15. A system for assisting transvaginal hysterectomy, said system comprising:
a frame positionable adjacent a vaginal os or a cervical os, said frame defining an opening for passing a mobilized uterus therethrough; and
coagulation and cutting elements positioned relative to the opening to engage structures which connect the uterus to supporting tissues as the mobilized uterus is drawn through the opening.

16. A system as in claim 15, further comprising a traction device coupled to the frame, said traction device being adapted to engage the mobilized uterus and advance the mobilized uterus through the opening.

17. A system as in claim 16, wherein the traction device comprises reciprocating grippers.

18. A system as in claim 17, wherein the reciprocating grippers have tissue engagement surfaces adapted to frictionally engage with tissue.

19. A system as in claim 18, wherein the tissue engagement surfaces are toothed.

20. A system as in claim 15, further comprising a tissue dissection blade disposed across the frame opening.

21. A system as in claim 15, wherein the opening and/or the cutting and coagulation elements are adjustable to separate to accommodate the width of the uterus as it passes therethrough.

22. A system as in claim 21, wherein the opening is defined by a split ring having halves which are biased closed but which move apart as the uterus is drawn therethrough.

23. A system as in claim 22, wherein at least one coagulation and cutting element is coupled to each ring half.

24. A system as to claim 15, wherein the coagulation and cutting elements comprises separate energy applying components and severing components, wherein the energy applying components are deposed to engage the connecting structures prior to said structures being engaged by the severing components.

25. A system as in claim 24, wherein the energy applying components are radiofrequency electrodes and the severing components are blades.

26. A system as in claim 25, wherein the elements are fixed with a flat surface.

27. A system as in claim 25, wherein the electrodes are cylindrical and tread-like and rotatably mounted on the frame.

28. A system as in claim 27, wherein the electrodes rotate in response to the uterus passing therethrough.

29. A system as in claim 27, further comprising a drive which rotates the electrodes to move the uterus therethrough.
